(19) United States
(12) Patent Application Publication
(10) Pub. No.: US 2010/0111981 A1
(43) Pub. Date: May 6, 2010

(54) SYNTHETIC MONODISPERSE HEMOZOIN CRYSTALS PREPARATION AND USES THEREOF

(75) Inventors: David Scott Bohle, Dorval (CA); Martin Olivier, St-Lambert (CA)

Correspondence Address: GOUDREAU GAGE DUBUC, 2000 MCGILL COLLEGE, SUITE 2200, MONTREAL, QC, H3A 3H3, CA

(73) Assignee: The Royal Institution for the Advancement of Learning/McGill University, Montreal, QC (CA)

(21) Appl. No.: 12/305,672

(22) PCT Filed: Jun. 22, 2007
(86) PCT No.: PCT/CA2007/001115

§ 371 Date: Dec. 19, 2008

Related U.S. Application Data
(60) Provisional application No. 60/805,487, filed on Jun. 22, 2006.

(51) Int. Cl. A61K 39/00; C07F 15/02; A61K 31/555; A61P 37/02

(52) U.S. Cl. 424/184.1; 548/402; 514/185

(57) ABSTRACT

A synthetic monodisperse hemozoin crystals preparation, compositions and methods of preparation thereof, are described. Also described are uses thereof, including use as an adjuvant, use in an immunogenic or vaccine composition, use for enhancing or inducing immunogenicity, and corresponding prevention or treatment of disease or infection.

### FIELD OF THE INVENTION

The invention relates to a heme composition and uses thereof, such as for modulating an immune response, and more particularly its use as an adjuvant, e.g., in a vaccine composition.

### BACKGROUND OF THE INVENTION

Hemozoin (HZ), a crystalline and brown pigment, is a metabolic byproduct from the digestion (detoxification) of heme molecules by Plasmodium parasites. The parasite cannot excrete the free heme and does not possess heme oxygenase to recover the iron and detoxify the heme; it thus aggregates the heme into an insoluble crystal, HZ (Slater, A. F. et al. Proc. Natl. Acad. Sci. USA 88: 325, 1991; Slater, A. F. Exp. Parasitol. 74: 362, 1992). Chemically, HZ is a dimer of iron (III) (protoporphyrin-IX), (PP-1×=protoporphyrin-IX) which corresponds to the anhydride of hematin, Fe(III)(PP-IX)(OH) (Pagola, S. et al. Nature 404: 307, 2000). It is profoundly insoluble and stable and consists of heme units dimerized through reciprocal iron-carboxylate bonds.

Vaccines are considered to be one of the most successful and cost-effective medical interventions against infectious diseases (Hilleman M. R., Vaccine 18: 1436-1447, 2000). A vaccine is used to evoke an antigen-specific effectors and memory immune response against a human pathogen, with minimal adverse reactions and it should lead to a specific long-term protection against this pathogen.

Traditional live anti-viral and anti-bacterial vaccines typically require no immunological adjuvants. Similarly, killed virus vaccines are generally much more immunogenic than attenuated pathogen or subunit protein vaccines and, like live anti-viral vaccines, can be effective with no adjuvant or with adjuvants that have limited ability to stimulate immune responses. Recently developed attenuated pathogen or subunit protein vaccines, while offering significant advantages over the traditional vaccines in terms of safety and cost of production, generally have limited immunogenicity compared to whole viruses. As a result, these vaccines typically require adjuvants with significant immunostimulatory capability to reach their full potential in preventing disease.

A vaccine adjuvant is more precisely a particulate, solid or soluble agent that increases the specific immune responses to an antigen. Vaccine adjuvants can enhance the immune response to vaccine antigens in various ways. When weak antigens are available, they are very useful for augmenting the immunogenicity of these molecules, thereby enhancing their vaccinal potency. They are also used to enhance the speed, vigor, and persistence of the immune response to a strong antigen. They can also modify the nature of the immune response. Depending on which adjuvant is used to stimulate a protective immune response, humoral or cell-mediated immunity can be selected. An adjuvant can modulate antibody specificity, as well as its quantity, isotype and subclass distribution. When used in direct contact with mucous membrane (e.g. Intranasal) it can effectively induce mucosal immunity. Adjuvants are also useful for potentiating the immune responses in immunologically immature, immunosuppressed or senescent individuals, acting as an immunological booster. Also, an adjuvant can effectively decrease the dose of antigen and/or the frequency of injection necessary to provide protection.

Adjuvants are immunomodulators that are typically non-covalently linked to antigens and are formulated to enhance the host immune responses. Some of these adjuvants are toxic, however, and can cause undesirable side effects, making them unsuitable for use in humans and many animals. Indeed, only few adjuvants are routinely used in human and veterinary vaccines. Also, currently available adjuvants and vaccines fail to induce a proper immune response capable of protecting against or treating certain infectious diseases, (e.g., HIV and HCV).

Therefore, there is a need for the development of vaccine adjuvants and immunogenic compositions with improved safety and efficacy and reduced side effects.

### SUMMARY OF THE INVENTION

The invention relates to a heme preparation or composition and uses thereof.

Accordingly, in an aspect, the present invention provides a synthetic monodisperse hemozoin crystals preparation.

In another aspect, the present invention provides a process for producing a synthetic monodisperse hemozoin crystals preparation comprising:
a) providing an iron(III) protoporphyrin-IX in an alkaline solution substantially free of oxygen,
b) adjusting the pH of the solution to an acidic pH, e.g., to a pH between about 3 and about 5, by slowly adding an acid,
c) incubating the solution under conditions permitting precipitation of hemozoin crystals, and
d) collecting the precipitated hemozoin crystals.

In an embodiment, the above-mentioned incubation is at a temperature between about 15° C. to about 80° C.

In an embodiment, the above-mentioned incubation is for a time period between about 4 hours to about 48 hours.

The present invention further provides a synthetic monodisperse hemozoin crystals preparation produced by the above-mentioned process.

In an embodiment, the majority of the crystals in said preparation have:
a) a length between about 0.8 μm to about 1.2 μm
b) a width between about 0.1 μm to about 0.2 μm;
c) a thickness between about 0.01 μm to about 0.15 μm; or
d) any combination of (a)-(c).

In an embodiment, the majority of the crystals in said preparation have:
a) a length of about 1 μm
b) a width of about 0.19 μm
c) a thickness of about 0.09 μm; or
d) any combination of (a)-(c).

In an embodiment, at least about 90% of the crystals in the above-mentioned preparation have:
a) a length of about 1 μm
b) a width of about 0.19 μm
c) a thickness of about 0.09 μm; or
d) any combination of (a)-(c).

In an embodiment, the above-mentioned iron(III) protoporphyrin-IX is hemin.

In an embodiment, in respect of the above-mentioned process, the pH of the solution is adjusted (step (b)) to between about 4.0 and 4.8. In a further embodiment, in respect of the above-mentioned process, the pH of the solution is adjusted (step (b)) to about 4.8.

In an embodiment, in respect of the above-mentioned process, the temperature (step (c)) is about 70° C.

In an embodiment, the above-mentioned acid is a carboxylic acid or an inorganic acid. In a further embodiment, the above-mentioned carboxylic acid is a liquid carboxylic acid. In a further embodiment, the above-mentioned liquid carboxylic acid is acetic acid or propionic acid. In yet a further embodiment, the above-mentioned carboxylic acid is propionic acid.

In an embodiment, in respect of the above-mentioned process, the acid is added over a period of about 20 minutes.

In an embodiment, in respect of the above-mentioned process, the time period is about 18 hours.

In another aspect, the invention provides an adjuvant composition comprising the above-mentioned preparation and a pharmaceutically acceptable excipient or carrier.

In another aspect, the invention further provides an immunogenic composition comprising the above-mentioned preparation, and one or more antigen(s).

In an embodiment of the above-mentioned immunogenic composition, at least one of the one or more antigen(s) is coated on the crystals.

In another aspect, the present invention provides a method for preventing or treating a microbial infection in an animal, comprising administering the above-mentioned immunogenic composition to said animal.

In another aspect, the present invention provides a method for preventing or treating a cancer in an animal, comprising administering the above-mentioned immunogenic composition to said animal.

In another aspect, the present invention provides a method for enhancing the immunogenicity of an antigen in an animal, comprising administering said antigen and the above-mentioned preparation to said animal.

In another aspect, the invention provides a package comprising the above-mentioned preparation or composition together with instructions for its use as an adjuvant.

In an embodiment, the above-mentioned antigen is admixed with the preparation.

In an embodiment, the above-mentioned antigen is coated on said crystals.

In an embodiment, the above-mentioned antigen is a polypeptide, a peptide, a polysaccharide, a lipid, a glycolipid, a phospholipid, a polynucleotide encoding the protein, a polynucleotide encoding the peptide, or a fragment of any of the foregoing.

In an embodiment, the above-mentioned antigen is a microbial antigen.

In an embodiment, the above-mentioned antigen is a tumour antigen.

In an embodiment, the above-mentioned antigen is derived from Leishmania.

In another aspect, the invention provides the use of the above-mentioned preparation for the preparation of a medicament.

In another aspect, the invention provides the use of the above-mentioned preparation as an adjuvant.

In another aspect, the invention provides a method for modulating the production of an inflammatory molecule in a biological system comprising contacting said biological system with the above-mentioned composition.

The invention further provides a method for modulating the production of an inflammatory molecule in an animal comprising administrating the above-mentioned composition to said animal.

The invention further provides the above-mentioned method, wherein the modulation is inducing or enhancing production of an inflammatory molecule.

The invention further provides the above-mentioned method, wherein said biological system is a cell.

The invention further provides the above-mentioned method, wherein said cell is an immune cell.

The invention further provides the above-mentioned method, wherein said cell is a monocyte/macrophage.

In an embodiment, the above-mentioned animal is a mammal.

In an embodiment, the above-mentioned mammal is a human.

In an embodiment, the above-mentioned inflammatory molecule is MIP-1β, MIP-1α, MIP-2, IP-10, MCP-1, IL-1α, IL-1β or IL-6.

### BRIEF DESCRIPTION OF THE DRAWINGS

The invention will now be described in greater detail having regard to the appended drawings, which relate to the following:

FIG. 1 shows a scanning electron microscopy (SEM) images of an A. amorphous hemozoin (aHZ), B. (inset) hemozoin from Plasmodium falciparum (PfHZ), C. synthetic Plasmodium like hemozoin (sPLHz) and D. synthetic slow crystallized hemozoin (scHZ);

FIG. 2 shows a X-ray powder diffraction patterns of three samples of synthetic Hemozoin corresponding to an amorphous hemozoin sample (A) and crystalline hemozoin samples (scHZ (B) and sPLHz (C));

FIG. 3 shows that hemozoin potentiates vaccination using soluble leishmania antigen (SLA). Mice (6-8 weeks old) received a single s.c. injection of SLA or SLA+HZ in a total volume of 100 ml. Two-weeks post inoculation, animals were challenged with infectious Leishmania donovani promastigotes (10⁷ parasite in 200 ml, i.v. injection). Two weeks later parasitic load was monitored, on a liver impression smear stained with giemsa, by counting the number of parasite per 1000 leukocyte nuclei counted. SLA/sPLHz vaccinated animals showed a significant protection against infectious parasite. Mice having received sPLHz only showed similar parasitic load as Nil (saline injected). SLA=Soluble Leishmania antigen; HZ=sPLHz;

FIG. 4 shows (A) a dose-response of HZ-induced macrophage chemokine gene expression, as measured by RNase protection assay. Values of gene expression are normalized over L32. Results in (B) represent the mean of 3 independent experiments. Grey bars=aHz; Black bars=sPLHz; Hatched bars=scHz;

FIG. 5 shows a time course of HZ-induced macrophage chemokine gene expression, as measured by RNase protection assay. Values of gene expression are normalized over L32. Results in (B) represent the mean of 3 independent experiments. Grey bars=aHZ; Black bars=sPLHz; Hatched bars=scHZ;

FIG. 6 shows the induction of liver cytokine (upper panel) and macrophage chemokine (lower panel) gene expression by Hz, as measured by RNase protection assay; and

FIG. 7 shows a gel analysis of serum proteins attaching to sPLHz (HZ). 100 μg of sPLHz was incubated with 200 μl of FBS/PBS at 37° C. for 1 h, then washed 3 times with PBS 1× and (A) resuspended in western sample loading buffer, then the samples were incubated 5 minutes at 95° C. and run on a 10% SDS-gel or (B) the samples were incubated in 2-D gel SDS buffer and run on a 1-D/2-D gel system. Proteins were revealed by silver staining.

### DETAILED DESCRIPTION OF THE INVENTION

In an aspect, the present invention relates to a synthetic monodisperse hemozoin crystals preparation and its use for the preparation of an adjuvant. The present invention also relates to a process for producing the above-mentioned synthetic monodisperse hemozoin crystals preparation. The preparation of present invention can be used to enhance the immunogenicity of a wide variety of antigens including, but not limited to, antigenic lipids, polypeptides and polynucleotides that encode antigenic polypeptides. The preparation of present invention can further be used in combination with other adjuvant formulations to further enhance the immunogenicity. Accordingly, the preparation of the present invention can be incorporated into a composition, e.g., an immunogenic, a vaccine or an immunomodulatory composition.

“Monodisperse” as used herein refers to a preparation comprising well-defined hemozoin (Hz) crystals, uniform with respect to crystal morphology, size, aspect ratio, and mosaicity. In particular, a monodisperse preparation has crystallites with a uniform set of morphologies, which are typically prismatic with an overall 10:2:1 ratio of principal dimensions (length:width:thickness). In addition to this uniformity in morphology and aspect ratio there is a similarity in the crystallite sizes in terms of either their length, width, or height (thickness).

“Immunogenic composition” or “vaccine” as used herein refers to a composition or formulation comprising one or more polypeptides or a vaccine vector. Vaccination methods for treating or preventing infection in a mammal comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g. via a patch).

The term “immunomodulatory” as used herein includes immunostimulatory as well as immunosuppressive effects. “Immunomodulation” generally refers to a qualitative and/or quantitative alteration in an overall immune response. For example, immunomodulation may refer to a shift towards a “Th1-type” immune response, as opposed to a “Th2-type” immune response, or the opposite.

“Adjuvant” refers to a substance which, when added to an immunogenic agent such as an antigen, nonspecifically enhances or potentiates an immune response to the agent in the host upon exposure to the mixture.

Treatment may be effected in a single dose or repeated at intervals. The appropriate dosage depends on various parameters understood by skilled artisans such as the vaccine or vaccine vector itself, the route of administration or the condition of the mammal to be vaccinated (weight, age and the like).

The composition of the present invention may be used for both prophylactic and therapeutic purposes. Accordingly, there is provided the use of synthetic monodisperse hemozoin crystals preparation in the manufacture of an immunogenic composition (e.g., a vaccine) for the prophylaxis and/or the treatment of viral, bacterial, fungal (e.g., Aspergillus), parasitic infections, allergy, cancer and other disorders. Accordingly, the present invention provides for a method of treating a mammal susceptible to or suffering from an infectious disease or cancer, or allergy, or autoimmune disease using the above-mentioned preparation or composition (e.g., by administering an effective amount of the preparation or composition to a subject). In a further aspect of the present invention, there is provided a vaccine or adjuvant combination, comprising synthetic monodisperse hemozoin crystals preparation, as herein described for use as a medicament. Immunogenic/vaccine preparation is generally described in New Trends and Developments in Vaccines, edited by Voller at al., University Park Press, Baltimore, Md., U.S.A. 1978.

An “effective amount” of a substance is that amount sufficient to effect beneficial or desired results, including clinical results, and, as such, an “effective amount” depends upon the context in which it is being applied. In the context of administering a preparation or composition of the present invention, an effective amount is, for example, an amount sufficient to achieve a modulation (quantitative and/or qualitative) of the immune response as compared to the immune response obtained when the antigen is administered alone. An effective amount can be administered in one or more administration(s).

As used herein, “treatment” is an approach for obtaining beneficial or desired results, including clinical results. For purposes of this invention, beneficial or desired clinical results include, but are not limited to, (i) prevention, that is, causing the clinical symptoms not to develop, e.g., preventing disease/infection from occurring and/or developing to a harmful state; (ii) alleviation or amelioration of one or more symptoms, (iii) diminishment of extent of disease, (iv) stabilizing (i.e., not worsening) state of disease, (v) preventing spread of disease, (vi) delay or slowing of disease progression, (vii) amelioration or palliation of the disease state, and (viii) remission (whether partial or total), whether detectable or undetectable. “Treatment” can also mean prolonging survival as compared to expected survival if not receiving treatment.

By “antigen” is meant a molecule that is capable of stimulating a host's immune system to make a cellular antigen-specific immune response and/or a humoral antibody response when the antigen is presented/administered. It refers to any natural or synthetic compound or chemical entity (lipids, phospholipids, glycolipids, saccharides, nucleic acids, etc.) capable of stimulating a immune response in a host. An antigen may contain one or more epitope(s). Normally, an epitope will include between about 3-15, generally about 5-15, amino acids. Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance (NMR). See, e.g., Epitope Mapping Protocols, supra. “Antigen” also refers to any natural or synthetic compound or chemical entity (lipids, phospholipids, glycolipids, saccharides, nucleic acids, etc.) capable of stimulating a immune response in a host.

The term “antigen” as used herein denotes both subunit antigens, i.e., antigens which are separate and discrete from a whole organism with which the antigen is associated in nature, as well as killed, attenuated or inactivated bacteria, viruses, parasites or other microbes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide that expresses an immunogenic protein, or antigenic determinant in vivo, such as in nucleic acid immunization applications, is also included in the definition of antigen herein. The antigenic polynucleotide can be delivered through two major routes, either using a viral or bacterial host as gene delivery vehicle (live vaccine vector) or administering the gene in a free form, e.g., inserted into a plasmid (DNA vaccine). Viral and bacterial vaccine vectors are well known in the art (see New Generation Vaccines, 3^{rd} edition, 2004 and Vaccine Protocols, 2^{nd} edition, Humana Press, 2003) and include, for example, Poxvirus, adenovirus, Measles virus, alphavirus, Yellow Fever virus, Semliki Forest virus, poliovirus, herpex simplex virus, vesicular stomatitis virus, Listeria monocytogenes, Salmonella and Shigella. The vaccine vector contains a polynucleotide antigen that is placed under the control of elements required for expression.

As used herein, the vaccine vector expresses one or several antigenic polypeptides or derivatives thereof. The vaccine vector may express additionally one or more immunomodulatory molecule(s), such as a co-stimulatory molecule (e.g., CD28, 4-1 BBL) or a cytokine (e.g., IL-2, IL-12), which enhances the immune response (adjuvant effect). It is understood that each of the components to be expressed is placed under the control of elements required for expression in a mammalian cell.

Further, for purposes of the present invention, antigens can be derived from any of several known viruses, bacteria, parasites and fungi, as well as any of the various tumor antigens.

Preferably the vaccine formulations of the present invention contain an antigen or antigenic composition capable of eliciting an immune response against a human pathogen, which antigen or antigenic composition is derived from Human Immunodeficiency virus (HIV), such as Tat, Nef, Gag, Pol, gp120 or gp160, human herpes viruses, such as gD or derivatives thereof or Immediate Early protein such as ICP27 from HSV1 or HSV2, cytomegalovirus ((esp Human) (such as gB or derivatives thereof), Rotavirus (including live-attenuated viruses), Epstein Barr virus (such as gp350 or derivatives thereof), Varicella Zoster Virus (such as gpI, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, . . . ), flaviviruses (e.g. Yellow Fever Virus, Dengue Virus, Tick-borne encephalitis virus, Japanese Encephalitis Virus) or Influenza virus (whole live or inactivated virus, split influenza virus, grown in eggs or MDCK cells, or whole flu virosomes (as described by R. Gluck, Vaccine, 1992, 10, 915-920) or purified or recombinant proteins thereof, such as HA, NP, NA, or M proteins, or combinations thereof).

Antigens can also be derived from bacterial pathogens such as Neisseria spp, including N. gonorrhea and N. meningitidis (for example capsular polysaccharides and conjugates thereof, transferrin-binding proteins, lactoferrin binding proteins, PiIC, adhesins); S. pyogenes (for example M proteins or fragments thereof, C5A protease, lipoteichoic acids), S. agalactiae, S. mutans: H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C, Th Ra12, Tb H9, Tb Ra35, Tb38-1, Erd 14, DPV, MTI, MSL, mTTC2 and hTCC1), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example, clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC. DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp. including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microtia; Trypanosoma spp., including T. cruzi; Giardia spp., including G. lamblia; Leishmania spp., including L. major, Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans, Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884), antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

The vaccine composition of the present invention may also comprise an anti-tumour antigen and be useful for the prevention or immunotherapeutic treatment of cancers. For example, the adjuvant formulation finds utility with tumour rejection antigens such as those for prostate, breast, colorectal, lung, pancreatic, renal or melanoma cancers. Exemplary antigens include MAGE 1, 3 and MAGE 4 or other MAGE antigens such as disclosed in WO 99/40188, PRAME, BAGE, Lage (also known as NY Eos 1) SAGE and HAGE (WO 99/53061) or GAGE (Robbins and Kawakami, 1996. Current Opinions in Immunology 8, pps 628-636; Van den Eynde et al., International Journal of Clinical & Laboratory Research 1997; 27(2):81-6; Correale et al. (1997), Journal of the National Cancer Institute 89, p293). Indeed these antigens are expressed in a wide range of tumour types such as melanoma, lung carcinoma, sarcoma and bladder carcinoma.

Other tumour-specific antigens are suitable for use in the vaccine composition of the present invention and include, but are not restricted to tumour-specific gangliosides such as GM2, and GM3 or conjugates thereof to carrier proteins; or said antigen may be a self peptide hormone such as whole length Gonadotrophin hormone releasing hormone (GnRH, WO 95/20600), a short 10 amino acid long peptide, useful in the treatment of many cancers, or in immunocastration.

Prostate antigens can also be utilised, such as Prostate specific antigen (PSA), PAP, STEAP, PSCA (PNAS 95(4) 1735-1740 1998), PSMA or Prostase (Ferguson et al., Proc. Natl. Acad. Sci. USA 1999. 96, 3114-3119).

Other tumour associated antigens (TAA) useful in the context of the present invention include: Carcinoembryonic antigen (CEA), KSA (also known as EpCAM), gp100, Plu-1 (J. Biol. Chem. 274 (22) 15633-15645, 1999), HASH-1, HasH-2, Cripto (Salomon et al., Bioessays 199, 21 61-70, U.S. Pat. No. 5,654,140) Criptin (U.S. Pat. No. 5,981,215). Additionally, antigens particularly relevant for vaccines in the therapy of cancer also comprise tyrosinase (Goldberg et al., Clin Cancer Res. 2005 Nov. 15; 11(22):8114-21; U.S. Patent application 20050142143) and survivin (Idenoue et al., Clin Cancer Res. 2005 Feb. 15; 11(4):1474-82; U.S. Patent Application 2004/0176573).

Mucin-derived peptides such as Muc1 (see for example U.S. Pat. Nos. 5,744,144, 5,827,666, WO 88/05054, U.S. Pat. No. 4,963,484). Specifically contemplated are Mud-derived peptides that comprise at least one repeat unit of the Muc1 peptide, preferably at least two such repeats and which is recognised by the SM3 antibody (U.S. Pat. No. 6,054,438). Other mucin-derived peptides include peptides from Muc5.

The present invention is also useful in combination with breast cancer antigens such as her2/Neu, mammaglobin (U.S. Pat. No. 5,668,267) or those disclosed in WO 00/52165, WO 99/33869, WO 99/19479, WO 98/45328. Her2/neu antigens are disclosed inter alia, in U.S. Pat. No. 5,801,005. Preferably the Her2/neu comprises the entire extracellular domain (comprising approximately amino acids 1-645) or fragments thereof and at least an immunogenic portion of or the entire intracellular domain approximately the C-terminal 580 amino acids. In particular, the intracellular portion should comprise the phosphorylation domain or fragments thereof. Such constructs are disclosed in WO 00/44899.

The compositions may comprise antigens associated with tumour-support mechanisms (e.g. angiogenesis, tumour invasion), for example Angiopoietin (Ang)-1 and -2, tyrosine kinase with immunoglobulin and epidermal growth factor homology domains (Tie)-2 as well as vascular endothelial growth factor (VEGF).

The vaccine or immunogenic composition of the present invention may be used for the prophylaxis or therapy of allergy. Such composition would comprise allergen specific (for example Der p1 and Der p5) and allergen non-specific antigens (for example peptides derived from human IgE, including but not restricted to the Stanworth decapeptide (EP 0477231 B1)). Other antigens include for example antigens derived from Aspergillus fumigatus (Asif A R, J Proteome Res. 2006 April; 5(4):954-62).

The vaccine or immunogenic composition of the present invention may also be used for the prophylaxis or therapy of chronic disorders others than allergy, cancer or infectious diseases. Such chronic disorders are diseases such as inflammatory and autoimmune diseases, atherosclerosis, and Alzheimer. Antigens relevant for the prophylaxis and the therapy of patients susceptible to or suffering from Alzheimer neurodegenerative disease are, in particular, the N-terminal 39-43 amino acid fragment (Abeta) of the amyloid precursor protein (APP) and smaller fragments. This antigen is disclosed in the International Patent Application No. WO 99/127944.

The composition of the present invention optionally comprises an emulsifying agent. A substantial number of suitable emulsifying agents (also referred to as surfactants or detergents) are used in the pharmaceutical sciences, any of which are typically the useful so long as they are sufficiently non-toxic. These include naturally derived materials such as gums from trees, vegetable protein, sugar-based polymers such as alginates and cellulose, and the like. Certain oxypolymers or polymers having a hydroxide or other hydrophilic substituent on the carbon backbone have surfactant activity, for example, povidone, polyvinyl alcohol, and glycol ether-based mono- and poly-functional compounds. Long chain fatty-acid-derived compounds form another substantial group of emulsifying agents that could be used in this invention. Specific examples of suitable emulsifying agents that can be used in accordance with the present invention include the following:

1. Water-soluble soaps, such as the sodium, potassium, ammonium and alkanol-ammonium salts of higher fatty acids (C₁₀-C₂₂), and, particularly sodium and potassium tallow and coconut soaps.

2. Anionic synthetic non-soap detergents, which can be represented by the water-soluble salts of organic sulfuric acid reaction products having in their molecular structure an alkyl radical containing from about 8 to 22 carbon atoms and a radical selected from the group consisting of sulfonic acid and sulfuric acid ester radicals. Examples of these are the sodium or potassium alkyl sulfates, derived from tallow or coconut oil; sodium or potassium alkyl benzene sulfonates; sodium alkyl glyceryl ether sulfonates; sodium coconut oil fatty acid monoglyceride sulfonates and sulfates; sodium or potassium salts of sulfuric acid esters of the reaction product of one mole of a higher fatty alcohol and about 1 to 6 moles of ethylene oxide; sodium or potassium alkyl phenol ethylene oxide ether sulfonates, with 1 to 10 units of ethylene oxide per molecule and in which the alkyl radicals contain from 8 to 12 carbon atoms; the reaction product of fatty acids esterified with isethionic acid and neutralized with sodium hydroxide; sodium or potassium salts of fatty acid amide of a methyl tauride; and sodium and potassium salts of SO₃-sulfonated C₁₀-C₂₄ alpha-olefins.

3. Nonionic synthetic detergents made by the condensation of alkylene oxide groups with an organic hydrophobic compound. Typical hydrophobic groups include condensation products of propylene oxide with propylene glycol, alkyl phenols, condensation product of propylene oxide and ethylene diamine, aliphatic alcohols having 8 to 22 carbon atoms, and amides of fatty acids.

4. Nonionic detergents, such as amine oxides, phosphine oxides and sulfoxides, having semipolar characteristics. Specific examples of long chain tertiary amine oxides include dimethyldodecylamine oxide and bis-(2-hydroxyethyl) dodecylamine. Specific examples of phosphine oxides are found in U.S. Pat. No. 3,304,263, and include dimethyldodecylphosphine oxide and dimethyl-(2-hydroxydodecyl) phosphine oxide.

5. Long chain sulfoxides, including those corresponding to the formula R₁—SO—R₂ wherein R₁ and R₂ are substituted or unsubstituted alkyl radicals, the former containing from about 10 to about 28 carbon atoms, whereas R₂ contains from 1 to 3 carbon atoms. Specific examples of these sulfoxides include dodecyl methyl sulfoxide and 3-hydroxy tridecyl methyl sulfoxide.

6. Ampholytic synthetic detergents, such as sodium 3-dodecylaminopropionate and sodium 3-dodecylaminopropane sulfonate.

7. Zwitterionic synthetic detergents, such as 3-(N,N-dimethyl-N-hexadecylammonio)propane-1-sulfonate and 3-(N,N-dimethyl-N-hexadecylammonio)-2-hydroxy propane-1-sulfonate.

Additionally, the following types of emulsifying agents can be used in a composition of the present invention: (a) soaps (i.e., alkali salts) of fatty acids, rosin acids, and tall oil; (b) alkyl arene sulfonates; (c) alkyl sulfates, including surfactants with both branched-chain and straight-chain hydrophobic groups, as well as primary and secondary sulfate groups; (d) sulfates and sulfonates containing an intermediate linkage between the hydrophobic and hydrophilic groups, such as the fatty acylated methyl taurides and the sulfated fatty monoglycerides; (e) long-chain acid esters of polyethylene glycol, especially the tall oil esters; (f) polyethylene glycol ethers of alkylphenols; (g) polyethylene glycol ethers of long-chain alcohols and mercaptans; and (h) fatty acyl diethanol amides. Since surfactants can be classified in more than one manner, a number of classes of surfactants set forth in this and other paragraphs overlap with one another.

The composition of the invention can be formulated into or with liposomes, preferably neutral or anionic liposomes, microspheres, ISCOMS, or virus-like-particles (VLPs) to facilitate delivery and/or enhance the immune response. These compounds are readily available to one skilled in the art; for example, see Liposomes: A Practical Approach, RCP New Ed, IRL press (1990).

The invention also relates to a pharmaceutical composition comprising a pharmaceutically acceptable carrier or excipient. As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. In particular, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound, use thereof in the pharmaceutical compositions of the invention is contemplated. Supplementary active compounds can also be incorporated into the compositions.

In another aspect, the present invention relates to a composition comprising hemozoin crystal preparation of the invention to modulate the inflammatory response, more particularly the secretion of inflammatory molecules/mediators, in a biological system (e.g., a cell, a tissue, an organ) or in an animal (e.g. a mammal, such as a human).

The expression “inflammatory molecules” or “inflammatory chemokines/cytokines” refers to molecules secreted by various cell types and whose functions include, for example, chemotaxis, integrin activation, cell differentiation, proliferation, secretion of mediators, and degranulation of distinct leukocyte subsets expressing specific receptors (Luster, A. D. 1998. N. Engl. J. Med. 338:436-445). As such, inflammatory chemokines/cytokines may play a role in autoimmune, allergic, and septic processes, as well as in the host response to infection, tumors, and vaccines. Inflammatory chemokines/cytokines include, for example, MCP-1/MCAF, MCP-2, MCP-3, MCP-4, Eotaxin, MIP-1 (3, MIP-1α, MIP-2, RANTES, TARC, MIP-3α, MDC/STCP-1, MPIF-2/Eotaxin-2, Eotaxin-3, MEC, GROα/MGSA-α, GROβ/MGSA-β, GROγ/MGSA-γ, IL-8, Mig, IP-10, I-TAC, Fractalkine, Lymphotactin, T cell activation protein-3 (TCA-3/CCL1/1-309), eotaxin, IL-1α, IL-1β, TNF-α, TNF-β, IL-2, IL-3, IL-4, IL-5, IL-6, IL-7, IL-9, IL-11, IL-12, IL-13, IL-15, IL-16, IL-17, IL-18, IL-21, IL-23, IL-25, IFN-γ, GM-CSF, SCF (Borish L C et al., J Allergy Clin Immunol. 2003 February; 111(2 Suppl):S460-75). In an embodiment, the above-mentioned inflammatory molecule is MIP-1β, MIP-1α, MIP-2, IP-10, MCP-1, IL-1β, IL-1β or IL-6. In an embodiment, the above-mentioned biological system is a cell (e.g. an immune or inflammatory cell). In a further embodiment, the above-mentioned cell is a monocyte/macrophage.

In another aspect, the present invention provides a process for producing a synthetic monodisperse hemozoin crystals preparation comprising:

a) providing an iron(III) protoporphyrin-IX in an alkaline solution substantially free of oxygen,

b) adjusting the pH of the solution to an acidic pH, e.g., to a pH between about 3 and about 5, by slowly adding an acid,

c) incubating the solution under conditions permitting precipitation of hemozoin crystals, and

d) collecting the precipitated hemozoin crystals.

In an embodiment, the above-mentioned incubation is at a temperature between about 15° C. to about 80° C.

In an embodiment, the above-mentioned incubation is for a time period between about 4 hours to about 48 hours.

In an embodiment, in respect of the above-mentioned process, the pH of the solution is adjusted (step (b)) to between about 4.0 and about 4.8. In a further embodiment, in respect of the above-mentioned process, the pH of the solution is adjusted (step (b)) to about 4.8.

In an embodiment, in respect of the above-mentioned process, the temperature (step (c)) is between about 50° C. to about 75° C. In a further embodiment, in respect of the above-mentioned process, the temperature (step (c)) is about 70° C.

In an embodiment, the above-mentioned acid is a carboxylic acid or an inorganic acid. In a further embodiment, the above-mentioned carboxylic acid is a liquid carboxylic acid. In a further embodiment, the above-mentioned liquid carboxylic acid is acetic acid or propionic acid. In yet a further embodiment, the above-mentioned carboxylic acid is propionic acid.

The present invention further provides a synthetic monodisperse hemozoin crystals preparation produced by the above-mentioned process.

In an embodiment, the majority of the crystals in said preparation have:

a) a length between about 0.8 μm to about 1.2 μm

b) a width between about 0.1 μm to about 0.2 μm;

c) a thickness between about 0.01 μm to about 0.15 μm; or

d) any combination of (a)-(c).

In an embodiment, the majority of the crystals in said preparation have:

a) a length of about 1 μm

b) a width of about 0.19 μm

c) thickness of about 0.09 μm; or

d) any combination of (a)-(c).

In an embodiment, at least about 50% of the crystals in the above-mentioned preparation have the above-mentioned length, width and thickness. In a further embodiment, at least about 60% of the crystals in the above-mentioned preparation have the above-mentioned length, width and thickness. In a further embodiment, at least about 70% of the crystals in the above-mentioned preparation have the above-mentioned length, width and thickness. In a further embodiment, at least about 80% of the crystals in the above-mentioned preparation have the above-mentioned length, width and thickness. In a further embodiment, at least about 90% of the crystals in the above-mentioned preparation have the above-mentioned length, width and thickness.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

Throughout this application, various references are referred to describe more fully the state of the art to which this invention pertains. The disclosures of these references are hereby incorporated by reference into the present disclosure.

The present invention is illustrated in further details by the following non-limiting examples.

### EXAMPLES

### Example 1

### Size Selective Preparations of Monodisperse Synthetic HZ Crystals

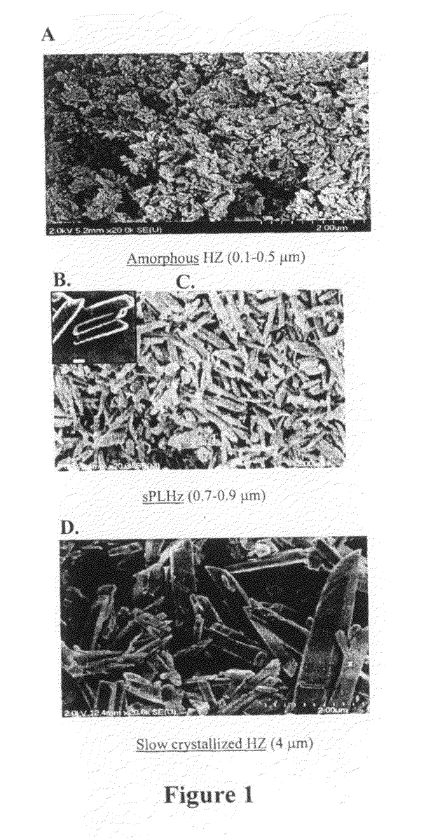

The methods described herein permit the production of a monodisperse hemozoin crystal preparation. “Monodisperse” as used herein refers to a preparation comprising well-defined hemozoin (HZ) crystals, uniform with respect to crystal morphology, size, aspect ratio, and mosaicity. In particular, a monodisperse preparation has crystallites with a uniform set of morphologies, which are typically prismatic with an overall 10:2:1 ratio of principal dimensions (length:width:thickness). In addition to this uniformity in morphology and aspect ratio there is a similarity in the crystallite sizes in terms of either their length, width, or height (thickness). Moreover all preparations return materials which are composed of chains of heme dimers, but whose characteristics may depend, for example, upon the initial conditions of solution pH and buffer, heme concentration, and solvent composition. As noted below, the synthetic monodisperse hemozoin crystal preparation prepared herein is sometimes referred to herein as “Synthetic Plasmodium Like Hemozoin” or “sPLHz”. A characteristic feature of the amorphous samples (FIG. 1A; 0.1-0.5 μm in size) is their high surface area, which have a random distribution of sites. Crystallite size (FIG. 1C (sPLHz; 1 μm), 1D (scHZ; 4 μm)), as well as batch homogeneity, are variables determined on a batch to batch basis. In the experimental protocols, amorphous and crystallite scHZ are used as controls, as well as Alum, a well-characterized adjuvant. Thus in aspects of size and morphology, these preparations are monodisperse (sPLHz and scHZ). Regardless of the preparative methods used, all samples are assessed for crystal size and homogeneity as described below. With these synthetic and analytical methodologies it is possible to control and assess the homogeneity of the crystal size as well as the morphology.

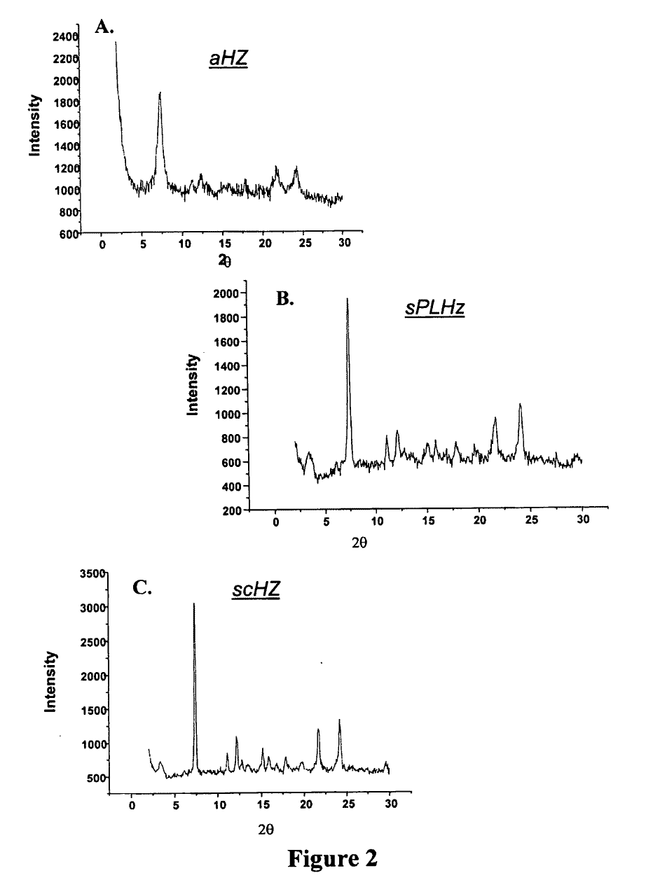

The starting material is an iron(III) protoporphyrin-IX, preferably high quality hemin, as for example the crystalline material from Fluke Chemical (Cat #51280, Tsutsui, K., Meth. Enzymol. 123, 331, (1986)). This starting material may be used without any further purification. However, prior to its use, it may be analyzed for homogeneity and purity by a combination of X-ray powder diffraction (see FIG. 2), IR and UV-Vis spectroscopy, as well as paramagnetic ¹H NMR spectroscopy by the Barbush method (Barbush, M. et al., Biochem Biophys Res Commun. 1985; 129(1): 70-5). For anaerobic and anhydrous preparations, a Vacuum Atmospheres Inert Atmosphere box may be employed with dried degassed solvents and reagents stored and utilized in an anhydrous atmosphere containing less than 1 ppm oxygen. For the preparation of solutions of hemin in water, the aqueous solution is preferably substantially free of oxygen, which can be obtained, for example, through degassing by dispersing an inert gas (e.g. nitrogen) into the solution before use. Once synthetic hemozoin (also named hematin anhydride) is obtained it is readily washed and purified with laboratory solvents with their equilibrium concentration of oxygen and water.

The term “synthetic” as used herein refers to products that are not directly obtained or isolated from natural sources.

### Example 2

### Methods of Assaying and Characterizing the New Preparations

The instant applicants have found that many known methods for characterizing scHZ are not practical for the determination of biological relevance. For example, while more common preparations of scHZ produce black precipitates that are insoluble in water and exhibit characteristic infrared bands at 1210 cm⁻¹, 1664 cm⁻¹, and 1711 cm⁻¹ corresponding to the propionic acid side chains, not all preparations produce monodisperse compositions with preferred uniform crystallinity. The following steps are used to confirm the physical specifications of the material.

Following IR spectroscopy and diffuse reflectance UV-Vis spectroscopy a dissolution assay is used to determine the heme content and verify gross contamination of insoluble phases. Powder diffraction is then performed to verify the unit cell composition, dispersity, and average crystallite size for the preparations. As a final check transmission electron microscopy (TEM) is used to verify all of these characteristics and to check for microscopic or amorphous contamination of the preparation. The reference point for these measurements are the prior results of the instant inventors (Bohle et al., 1994, Inorganic and Organometallic Polymers II, ACS Symposium Series (572) 497-515; Bohle et al., Biochem Biophys Res Commun. 1993 Jun. 15; 193(2):504-8.; Pagola et al., Nature. 2000 Mar. 16; 404(6775):307-10.; Bohle et al., Biochem Biophys Res Commun. 2002 May 31; 294(1):132-5.; Bohle, 1998, J. Am. Chem. Soc. 120: 8255-8256).

Amorphous Hemozoin (aHz)

Completely amorphous material is obtained by rapidly lowering the pH of a concentrated solution of hemin. For example using the anaerobic methods described above, 50 mL of a 150 mM solution of hemin in NaOH (0.1M) is allowed to stand for two hours after which 100 mL of propionic acid is added dropwise. It can also be obtained by the rapid stirring or rapid addition of propionic acid to the stock solution. In general aHz is a major contaminant in almost all previously reported preparations of hemozoin. It can also result from insufficient washing with NaHCO₃ in the later steps of the acid treated preparations. aHz is readily recognized by broadened peaks in the infrared spectrum, its the X-ray powder diffraction pattern and by poorly defined precipitates under SEM or TEM examination as illustrated in FIGS. 1 and 2.

Synthetic Plasmodium Like Hemozoin (sPLHz)

100 mL of a 0.1 M solution of NaOH is thoroughly degassed by bubbling nitrogen for 1 hour before hemin (0.8 mmol, 521.6 mg) is added and allowed to dissolve for 30 minutes with magnetic stirring, under a stream of nitrogen; the flask is wrapped in aluminum foil. The solution is adjusted to pH 4.8 by slow addition of acid, for example by dropwise addition of 4 mL of propionic acid over 20 minutes using a syringe pump. The flask is transferred to an oil bath at 70° C. and allowed to anneal for 18 hours with mild magnetic stirring. The black crystals can be observed at the bottom of the flask while the solution becomes completely clear. The mixture is transferred into 8×50 mL centrifuge tubes and centrifuged at 7000 rpm for 1 hour after which the clear supernatant is decanted. The crystals are then washed 3×3 hours with NaHCO₃ (0.1 M, 25 mL in each tube) with vortex stirring. Each time, the liquid is decanted after centrifugation for 1 hour at 7000 rpm. Each NaHCO₃ wash is intercalated with a water rinse or if the crystals are left overnight, they are left soaking in a 1:1 solution of H₂O:MeOH. Finally, the washes are supplemented with 3 washes with MilliQ H₂O and 3 washes of MeOH alternatively, slowly concentrating the crystals into 1×50 mL centrifuge tube. The resulting crystals are then dried thoroughly in a vacuum oven overnight over phosphorus pentoxide. This method yields a preparation of monodisperse HZ crystals, in which the majority of the crystals have a characteristic size: 1 μm length, 0.19 μm width and 0.09 μm thickness as determined by Field Emission Gun Scanning Electron Microscopy (FEG-SEM). These results represent averages of three fields with circa 50 crystals.

Synthetic Crystalline Hemozoin (scHZ)

A flask containing Hemin (0.8 mmol, 521.6 mg) was transferred to an inert atmosphere box and treated with 2,6-lutidine (20 mL) to dissolve it completely. The solution was further diluted with 100 mL of a 1:1 solution of methanol and dimethylsulfoxide. The flask was then sealed, wrapped with aluminum foil, removed from the inert box and allowed to stand undisturbed from 2 weeks to 15 months, depending on the desired yield. The flask was then unsealed and the black mixture was centrifuged at 7000 rpm for 1 hour. The supernatant was then decanted. The crystals were then washed once with NaHCO₃ (0.1 M) for 3 hours. Washes were completed by alternating distilled H₂O and MeOH 3 times. The sample was dried in a vacuum oven at 105° C. for 24 hrs.

This method yielded crystals of characteristic size: 4 μm length, 0.3 μm width and 0.1 μm thickness as determined by TEM and FEG-SEM.

### Example 3

### Use of sPLHz in Combination with Soluble Leishmania Antigen (SLA) to Test the Adjuvant Potential of Our New Synthetic Hz in a Leishmania Vaccination Model

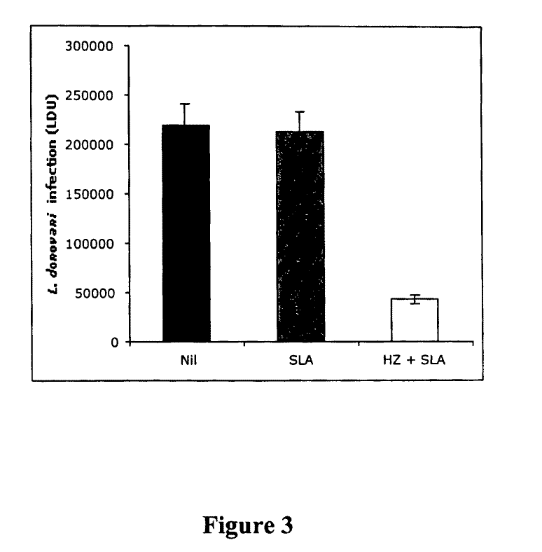

The results of these experiments are shown in FIG. 3. sPLHz (50 μg) was mixed with 100 μg of SLA prepared from Leishmania donovani promastigotes subjected to freeze/thawed disruption in endotoxin-free PBS, and injected sub-cutaneously in a final volume of 100 μl. Thereafter, two weeks post-inoculation, mice were challenged with infectious Leishmania donovani promastigotes (10⁷ parasites in 200 μl, i.v. injection). Two-weeks later, animals were anesthetized and their liver was removed, weighed and the main lobe cut by the center and used to make impression smear on a glass slide. The preparations were giemsa-stained using the Diff-Quick™ system. Parasitic load was obtained by counting the number of amastigote parasite form per 1000 leukocyte nuclei counted and by multiplying the number of amastigotes by the liver weight in mg. The value obtained is referred to as Leishmania donovani unit (LDU). The data represents the mean of 5 animals per group. As shown in FIG. 3, animals administered with saline or SLA alone showed similar level of infection whereas animals that received sPLHz+SLA showed a marked reduction of their parasitic load.

### Example 4

### Induction of Chemokine Gene Expression In Vitro and In Vivo by sPLHz

Chemokine mRNA expression was monitored using an RPA kit (mCK-5 RiboQuant™; BD PharMingen, San Diego Calif.), as previously described (Matte, C., and M. Olivier. 2002. J Infect Dis. 2002 Mar. 1; 185(5):673-81), to enable simultaneous detection of a large number of these inflammatory molecules (Lymphotactin, RANTES, MIP-1β, MIP-1α, MIP-2, IP-10, T cell activation protein [TCA]-3, and eotaxin). Total RNA was extracted from the stimulated and non-stimulated macrophages with TRizol™ (Life Technologies) according to the manufacturer's protocol. The commercial multiprobe was labelled with [a-³²P]dUTP using T7 RNA polymerase. Labelled probe (3′, 105 cpm) was added to 10 mg of total RNA, and allowed to hybridize for 16 h at 56° C. Resulting mRNA probe hybrids were subjected to an RNase A treatment, and extracted with phenol-chloroform. Protected hybrids were loaded on a 5% denaturing polyacrylamide sequencing gel. Once dried, the gel was exposed to a radiographic film at −80° C., and also subjected to densitometry analysis using a Molecular Imager FX and the analysis software Quantity One 1D™ version 4.4 (Bio-Rad). Chemokine density values were normalized to the housekeeping gene mouse ribosomal protein L32 (mL32), also present in the multiprobe template.

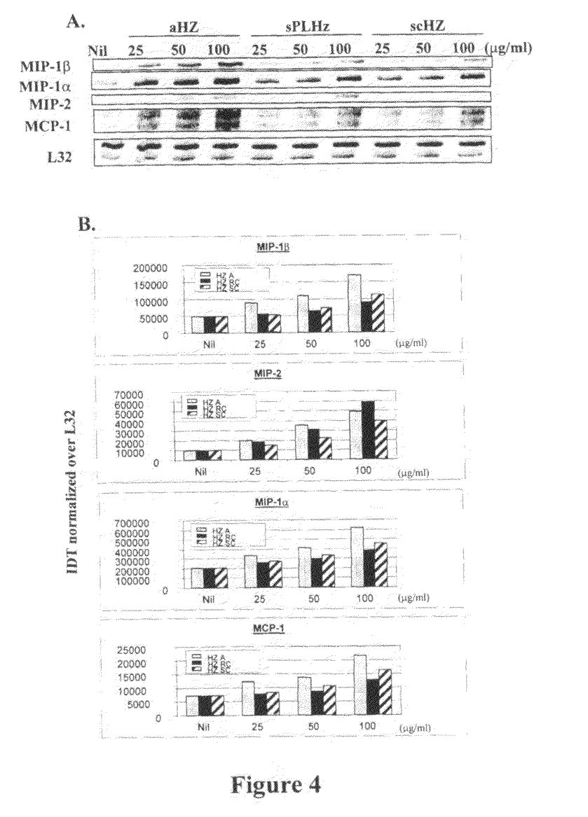

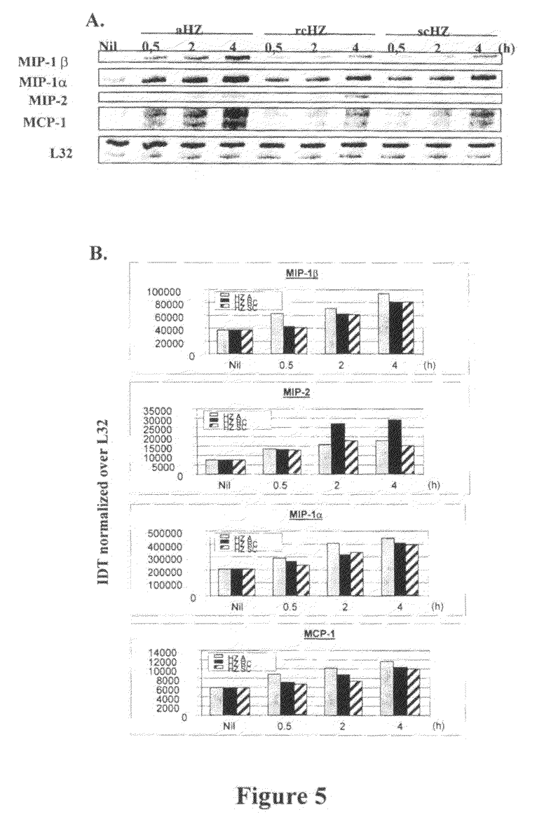

HZ-induced macrophage chemokine gene expression was measured by RNase protection assay (FIG. 4). 25-100 mg/ml of the various HZ was used to stimulate murine macrophages for a 2 hr period. sPLHz (HZ RC) and slow crystalline HZ (scHZ) similarly induced the majority of chemokine gene expression in a dose-dependent manner (A), whereas amorphous HZ (aHZ) seems to be a slightly stronger inducer for some chemokines (B). However sPLHz seems to be better at inducing MIP-2 chemokine (see time course FIG. 5). Values of gene expression are normalized over the expression of mL32. Results in (B) represent the mean of 3 independent experiments.

FIG. 5 shows a time-course of HZ-induced macrophage chemokine gene expression. Each preparation of HZ was used at a concentration of 50 μg/ml and stimulated over a 4 hr period. As measured by RNase protection assay, sPLHz (HZ RC) and slow (scHZ) crystalline HZ similarly induce the majority of chemokine gene expression in a time-dependent manner (A), whereas amorphous HZ (aHZ) seems to be a slightly stronger inducer for some chemokines, whereas sPLHz was a stronger inducer of MIP-2 ((B), average of 3 independent experiments). Values of gene expression are normalized over L32.

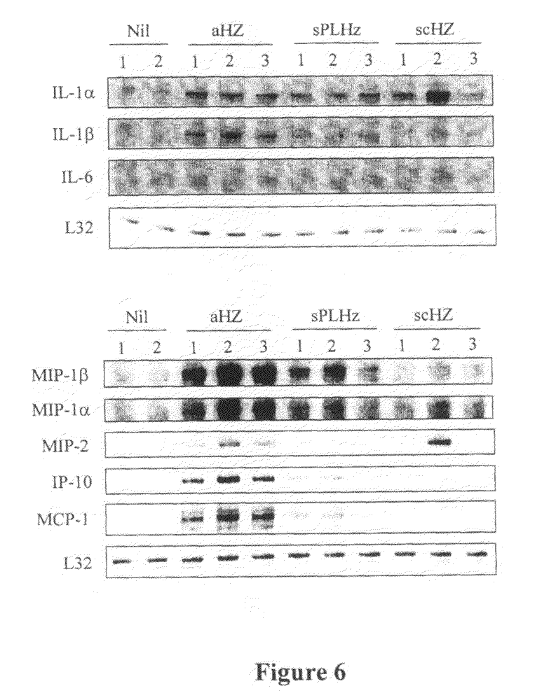

HZ-induced liver cytokine and chemokine gene expression was monitored (FIG. 6). As measured by RNase protection assay, sPLHz (HZ RC) and amorphous HZ (aHZ) similarly induce the in vivo expression of the majority of pro-inflammatory cytokines and chemokines tested (IL-1α, IL-1β and IL-6) (250 μg for 6 hr, i.v. injection in 200 μl endotoxin-free PBS). However, aHZ seems to cause stronger activation compared to sPLHz, which demonstrates that subtle differences may confer different levels of inflammation. Importantly, the slow crystalline HZ (scHZ) seems to be a less potent inducer of some cytokines and chemokines; suggesting that effectively the structure may differentiate the capacity of different preparations to induce immune and inflammatory responses.

Thus, the nature of the crystal, such as the monodisperse sizes and morphologies, plays a role in immune response. The sPLHz preparation described herein is capable of inducing a controlled inflammatory process. Heterodisperse preparations such as aHZ may manifest inadequate or too strong inflammatory events. Mixtures or preparations with very different crystal sizes result in particles with very different capacity to induce pro-inflammatory events and significant batch-to-batch variations. Thus, the more homogenous monodisperse preparation obtained with the preparation of the sPLHz described herein is under more strictly defined conditions, and provides a more predictable and sustainable inflammatory response.

### Example 5

### Adsorption of Antigens onto sPLHz and its Use as Coatable-Material or Vehicle To Deliver Antigens in Vaccinology

The use of sPLHz as a vehicle to administer potential antigens (e.g., for immunization) was then assessed. Thus, loading or adhesion of known bacterial antigens onto sPLHz could augment its modulator characteristics.

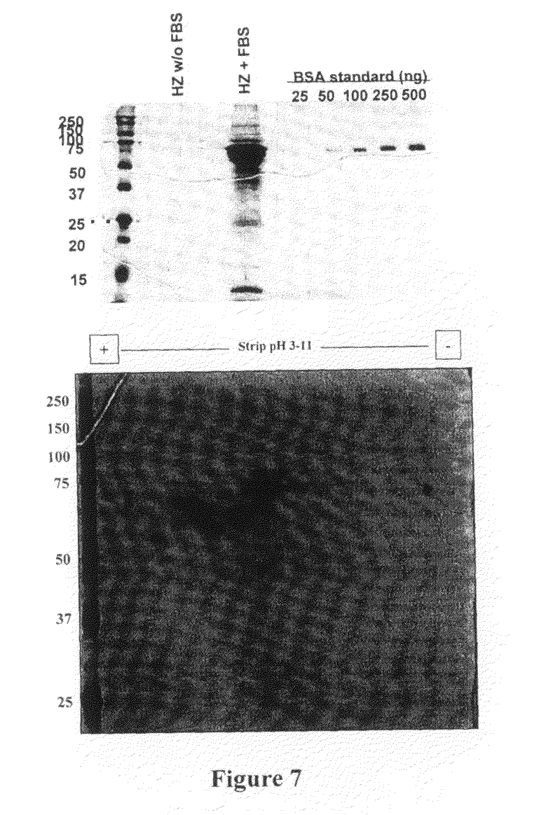

As shown in FIG. 7, sPLHz (100 μg) was incubated for 1 hr at 37° C. in RPMI medium in the presence or absence of 10% fetal bovine serum (FBS) (incubation has also been performed for 5 to 60 min, and all showed similar rapid attachment of host protein to the crystal). Thereafter, the material was washed in endotoxin-free PBS at least 3 times (washing of up to 8 times was also performed and the protein still adhered to the crystal) by spinning down the crystal with a high-speed microfuge. Finally, the host material still attached to the crystal was resuspended in 50 μl of SDS-PAGE sample loading buffer (SLB), heated for 5 min at 95° C. and spun down at maximal speed of the microfuge for 1 min. The supernatant (40 μl) was loaded on 10% SDS-PAGE gel and run at 200 V. After migration, the gel was fixed overnight in MeOH, acetic acid and water, and silver stained according to well-known commercial protocols.

As shown in FIG. 7, the sPLHz can be coated with a significant amount of protein onto its surface, at least up to 500 ng of material per 80 μg of sPLHz, and therefore may be used as vehicle to deliver an antigen of interest in a vaccine.

Table I shows the proteins from FBS bound to sPLHz as determined by MALDI-TOF MS/MS. One hundred (100) μg of sPLHz was incubated with 200 μl of FBS/PBS at 37° C. for 1 h, then washed 3-5 times with PBS 1× and resuspended in 15 μl of highly pure, RNAse/DNAse free water, and analyzed by MALDI-TOF MS/MS.

TABLE I
FBS proteins binding to
sPLHz

Albumin
α-2-macroglobulin precursor
α-2-HS glycoprotein
α-1-antitrypsin
α-fetoprotein
Angiotensinogen (precursor)
Apolipoprotein A-I precursor
Apolipoprotein A-II
Apolipoprotein C-II
Beta-2-glycoprotein
Bone derived growth factor
Complement C3
Complement C4
Fetuin
GCN
Gelsolin
Globin (α, β, γ and ε)
Hemoglobin (α, β, δ and γ)
Inter-alpha inhibitor (H2
polypeptide)
Osteoblast-specific factor 2
PEDF
serine/cysteine inhibitor (clade
A members 1 and 5, clade F)

Although the present invention has been described hereinabove by way of specific embodiments thereof, it can be modified, without departing from the spirit and nature of the subject invention as defined in the appended claims.

What is claimed is:

1. A synthetic monodisperse hemozoin crystals preparation.

2. A process for producing a synthetic monodisperse hemozoin crystals preparation comprising:
(a) providing an iron(III) protoporphyrin-IX in an alkaline solution substantially free of oxygen;
(b) adjusting the pH of the solution to between about 3 and about 5 by slowly adding an acid;
(c) incubating the solution under conditions permitting precipitation of hemozoin crystals; and
(d) collecting the precipitated hemozoin crystals.

3. The process of claim 2, wherein said incubation is at a temperature between about 15° C. and about 80° C.

4. The process of any one of claims 2-3, wherein said incubation is for a time period between about 4 hours and about 48 hours.

5. A synthetic monodisperse hemozoin crystals preparation produced by the process of any one of claims 2-4.

6. The preparation of claim 1 or 5, wherein the majority of the crystals in said preparation have:
(a) a length between about 0.8 μm and 1.2 μm;
(b) a width between about 0.1 μm and 0.2 μm;
(c) a thickness between about 0.01 μm and 0.15 μm; or
(d) any combination of (a)-(c).

7. The preparation of claim 6, wherein the majority of the crystals in said preparation have:
(a) a length of about 1 μm;
(b) a width of about 0.19 μm;
(c) a thickness of about 0.09 μm; or
(d) any combination of (a)-(c).

8. The preparation of claim 1 or 5, wherein at least about 90% of the crystals in said preparation have:
(a) a length between about 0.8 μm and 1.2 μm;
(b) a width between about 0.1 μm and 0.2 μm;
(c) a thickness between about 0.01 μm and 0.15 μm; or
(d) any combination of (a)-(c).

9. The preparation of claim 8, wherein at least about 90% of the crystals in said preparation have:
(a) a length of about 1.0 μm;
(b) a width of about 0.19;
(c) a thickness of about 0.09 μm; or
(d) any combination of (a)-(c).

10. The process of any one of claims 2-4, wherein said iron(III) protoporphyrin-IX is hemin.

11. The process of any one of claims 2-4 and 9-10, wherein said pH is about 4.8.

12. The process of any one of claims 2-4 and 9-11, wherein said temperature is about 70° C.

13. The process of any one of claims 2-4 and 9-12, wherein said acid is a liquid carboxylic acid.

14. The process of claim 13, wherein said liquid carboxylic acid is acetic acid or propionic acid.

15. The process of claim 14, wherein said liquid carboxylic acid is propionic acid.

16. The process of any one of claims 2-4 and 9-15, wherein said acid is added over a period of about 20 minutes.

17. The process of any one of claims 2-4 and 9-16, wherein said time period is about 18 hours.

18. An adjuvant composition comprising the preparation of any one of claims 1, 5 and 8, and a pharmaceutically acceptable excipient or carrier.

19. An immunogenic composition comprising the preparation of any one of claims 1, 5 and 8, and one or more antigen(s).

20. The immunogenic composition of claim 19, wherein at least one of the one or more antigen(s) is coated on the crystals.

21. The immunogenic composition of claim 19 or 20, further comprising a pharmaceutically acceptable excipient or carrier.

22. The immunogenic composition of any one of claims 19-21, wherein said antigen is a polypeptide, a peptide, a polysaccharide, a lipid, a glycolipid, a phospholipid, a polynucleotide encoding the protein, a polynucleotide encoding the peptide, or a fragment of any of the foregoing.

23. The immunogenic composition of any one of claims 19-21, wherein said antigen is a microbial antigen.

24. The immunogenic composition of any one of claims 19-21, wherein said antigen is a tumour antigen.

25. The immunogenic composition of claim 23, wherein said antigen is derived from Leishmania.

26. A method for preventing or treating a microbial infection in an animal, comprising administering the immunogenic composition of claim 23 to said animal.

27. A method for preventing or treating a cancer in an animal, comprising administering the immunogenic composition of claim 24 to said animal.

28. A method for enhancing the immunogenicity of an antigen in an animal, comprising administering said antigen and the preparation of any one of claims 1, 5 and 8, or the composition of claim 18, to said animal.

29. The method of claim 28, wherein the antigen is admixed with the preparation.

30. The method of any one of claims 26 to 29, wherein said animal is a mammal.

31. The method of claim 30, wherein said mammal is a human.

32. Use of the preparation of any one of claims 1, 5 and 8 for the preparation of a medicament.

33. Use of the preparation of any one of claims 1, 5 and 8 as an adjuvant.

34. The use of claim 32, wherein said medicament is a vaccine or an immunomodulatory agent.

35. A package comprising the preparation of any one of claims 1, 5 and 8, or the adjuvant composition of claim 18, together with instructions for its use as an adjuvant.

36. The package of claim 35, further comprising an antigen.

37. The package of claim 36, wherein the preparation is admixed with the antigen.

38. The package of claim 36 or 37, wherein the crystals in the preparation are coated with the antigen.

39. A method for modulating the production of an inflammatory molecule in a biological system comprising contacting said biological system with the preparation of any one of claims 1, 5 and 8 or the composition of claim 18.

40. A method for modulating the production of an inflammatory molecule in an animal comprising administrating the preparation of any one of claims 1, 5 and 8 or the composition of claim 18 to said animal.

41. The method of claim 39 or 40, wherein the modulation is inducing or enhancing the production of an inflammatory molecule.

42. The method of claim 39, wherein said biological system is a cell.

43. The method of claim 42, wherein said cell is an immune cell.

44. The method of claim 43, wherein said immune cell is a macrophage or a monocyte.

45. The method of claim 40, wherein said animal is a mammal.

46. The method of claim 45, wherein said mammal is a human.

47. The method of any one of claims 39-46, wherein said inflammatory molecule is MIP-1β, MIP-1α, MIP-2, IP-10, MCP-1, IL-1α, IL-1β or IL-6.
